(19) United States
(12) Patent Application Publication
(10) Pub. No.: US 2011/0256231 A1
(43) Pub. Date: Oct. 20, 2011

(54) METHOD FOR PREPARING A MICRO-PARTICLE DRUG IN HEMISPHERE-SHAPED DOSAGE FORM AND APPLICATIONS THEREOF

(76) Inventors: Yung-Sheng Lin, Hsinchu (TW); Chun-Ting Lin, Hsinchu (TW); Keng-Shiang Huang, Hsinchu (TW); Chih-Hui Yang, Hsinchu (TW); Sheng-Yi Hsiao, Hsinchu (TW); Chih-Cheng Huang, Hsinchu (TW); Shih-Feng Tseng, Hsinchu (TW)

(21) Appl. No.: 12/763,408

(22) Filed: Apr. 20, 2010

(51) Int. Cl. A61K 9/14

(52) U.S. Cl. 424/499; 427/2.14

(57) ABSTRACT

A method for preparing drug in hemisphere-shaped dosage form. A high molecular weight solution containing the drug is prepared, and the solution is then dropped on a base material. The interface phenomena between the solution and different base materials makes the drop of high molecular weight solution containing the drug form a hemisphere-shape. After solidifying by cross-link or evaporation, the drug in hemisphere-shaped dosage form is obtained. The advantages of the preparation method are a simple and fast process, and simple operation. Applications of the method to prepare a drug in hemisphere-shaped dosage form are also provided.

### BACKGROUND OF THE INVENTION

1. Field of the Invention

The present invention is related to a novel method of preparing a drug delivery form, in particular, a method of preparing a micro-particle drug in hemisphere-shaped dosage form, and applications thereof.

2. Description of the Prior Art

Recent studies pertain to Drug Delivery Systems (DDS), i.e., preventing overdosage, side effects, promoting safe and effective ways for micro-particle drug delivery, and providing the necessary minimal amount of a drug dose to the necessary part in the necessary time. Most studies focus on development of Drug Delivery Systems for 1) slowly dissolving a drug in the body, and/or 2) delivering an adequate amount of the drug by the blood stream to the nidus for treatment. These studies relate to the improvement of a drug's characteristics or the development of materials, methods or structures for embedding the drug. In 1998, Ozdemir and Karatas pointed out that the shape or geometry of the drug could affect the release rate thereof. They also found that a water-soluble drug or slightly oil-soluble drug in a hemisphere shape achieves a certain release rate in comparison with that of the drug in other shapes, such as a cylinder, slab, or biconvex form. In “Analysis of nano drug carriers towards optimum release rate” (Journal of Medical Engineering & Technology, Vol. 31, No. 4, pp. 243-252) Ng et al. indicate that a drug in hemisphere-shaped dosage form could reach a zero-order drug release rate in comparison to tetrahedron, cylinder, sphere, cuboid, and octahedron shapes. However, the art remains silent with regard to preparing and/or applying a hemisphere-shaped dosage forms for drug delivery.

Besides improving the release rate of a drug, the development of a new dosage form for drug delivery can reduce the research and development costs of a drug greatly, thus giving the product itself a high added-value. In comparison with development of a new drug, the risk of developing a new dosage form is much lower. Based on the importance of developing new dosage forms for drug delivery, the inventors of the present invention provide a method for preparing a micro-particle drug in a hemisphere-shaped dosage form and applications thereof.

### SUMMARY OF THE INVENTION

The object of the present invention is to provide a novel method for preparing a micro-particle drug in a hemisphere-shaped dosage form.

Another purpose of the present invention is to provide applications of the method for preparing a micro-particle drug in a hemisphere-shaped dosage form.

In order to accomplish the purposes listed above, the method of the present invention comprises preparing a high molecular weight solution containing the drug and the solution and then dropping this solution on a base material. The interface phenomena between the drug solution and different base materials makes the drug solution form a hemisphere shape. After solidifying and separating the drug solution, a hemisphere-shaped dosage form is obtained. The drug in hemisphere-shaped dosage form contains drug, high molecular weight ingredient and water, and thus can achieve a zero-order drug release rate in a patient's body.

These features and advantages of the present invention will be fully understood and appreciated from the following detailed description and the accompanying drawings.

### BRIEF DESCRIPTION OF THE DRAWINGS

FIGS. 1A-1C demonstrate the preparation of a hemisphere-shaped dosage form of a drug.

FIGS. 2A and 2B show the operation of the invention.

### DETAILED DESCRIPTION OF THE PREFERRED EMBODIMENTS

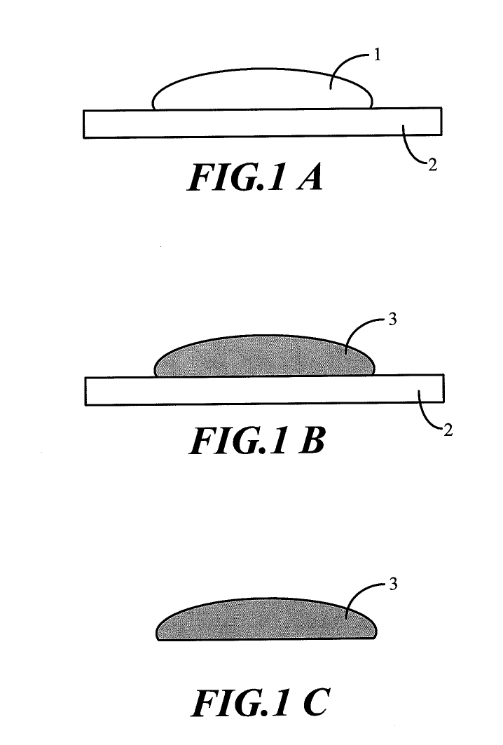

The present invention provides a novel method for preparation of a hemisphere-shaped dosage form of a drug. As shown in FIGS. 1A-1C, the method comprises preparing a high molecular weight solution containing a drug and dropping the solution on a base material 2 to produce a hemisphere-shaped drop of drug solution 1. After solidifying and separating the solution from the base material 2, the drug in a hemisphere-shaped dosage form 3 is obtained.

Solidifying steps include, but are not limited to, cross-link, evaporation, and so on.

Separation steps include, but are not limited to, methods of physical or chemical separation and any separation methods that are suitable to be used in this invention.

The drugs include, but are not limited to, any drugs that are suitable to be used in the preparation of this invention.

The high molecular weight ingredient of the high molecular solutions include, but are not limited to, alginate, chitin, chitosan, poly lactic acid (PLA), poly glycolic acid (PGA) and any high molecular ingredients that are suitable to prepare a high molecular weight solution of this invention

In one preferred embodiment, the high molecular weight solution contains at least 1%˜5% drug, 1%˜40% alginate, and 55%˜98% water. Furthermore, the high-molecular-weight solution is cross-linked by an effective amount of calcium ion (e.g. a compound or composition that contains calcium), and a solidified micro-particle of alginate in a hemisphere-shaped dosage form is prepared.

The “effective amount” of a calcium ion (e.g., a compound or composition that contains calcium) means that the amount or concentration of calcium ion is enough to trigger cross-linking and solidifying of the high molecular weight solution.

In another preferred embodiment, the high molecular weight solution contains at least 0.05%˜20% drug, 1.95%˜49.95% PLA, and 50%˜98% organic solvent, wherein the organic solvent includes, but is not limited to, methylene chloride, ethyl acetate, and acetone. Furthermore, the solidified micro-particle of PLA in a hemisphere-shaped dosage form is prepared after complete evaporation of the organic solvent.

The size of the micro-particle drug in a hemisphere-shaped dosage form prepared by the present invention could be micron-scale or nano-scale.

The invention will be illustrated with the examples as follows, without the intention that the invention is limited thereto. The substance or material herein are easily obtained, and the source of materials is not limited to following examples.

### EXAMPLE 1

### Preparation of Micro-Particle of Alginate in Hemi-Sphere Dosage Form

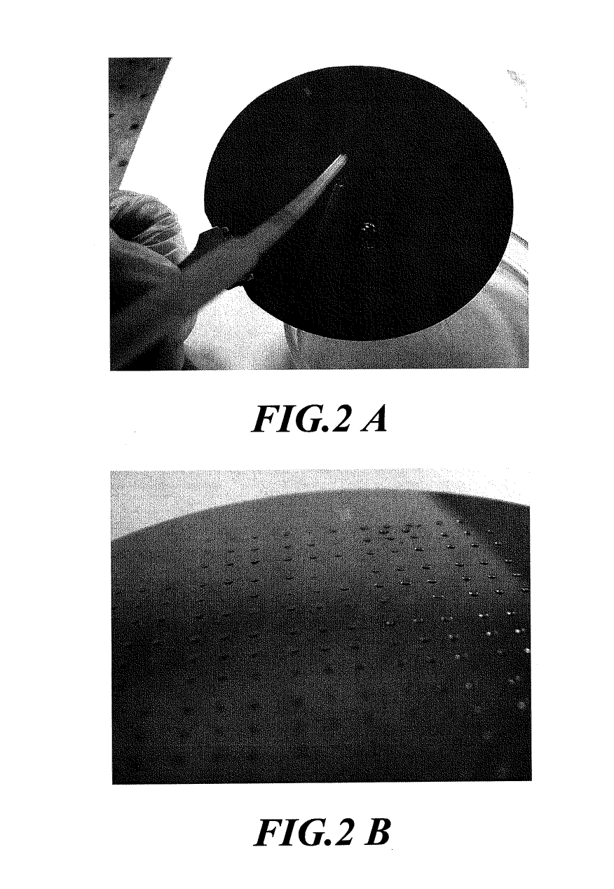

Please refer to FIGS. 1A-1C and FIGS. 2A-2B for the following description. An aqueous solution containing drug and alginate is prepared, and the solution is then dripped on a base material to form a hemisphere-shaped aqueous drop. A solution containing calcium is then added into the hemisphere-shaped aqueous drop to trigger the cross-link reaction (i.e., the solidification step). The alginate is gelatinous and the hemisphere-shaped micro-particle is then formed. The base material is removed to obtain a micro-particle drug with alginate in a hemisphere-shaped dosage form.

### EXAMPLE 2

### Preparation of Micro-Particle of PLA in Hemi-Sphere Dosage Form

A methylene chloride solution containing drug and PLA is prepared, and the solution is then dripped on a base material. A hemisphere-shaped drop of the organic solution containing drug and PLA is formed and then stewed for a while to allow the organic solvent to evaporate, after which the hemisphere-shaped drop is semi-solidified and not mobile. The base material is then removed to obtain the semi-solidified micro-particle drug with PLA in a hemisphere-shaped dosage form. Finally, the solidified micro-particle drug with PLA in a hemisphere-shaped dosage form is obtained by further evaporation to remove any remaining organic solvent completely. Said solidified micro-particle drug with PLA contains 0.1%˜40% of drug and 60%˜99.9% of PLA.

Compared with the current state of the arts and products, the present invention provides a preparation method and its applications to produce a micro-particle drug in hemisphere-shaped dosage form. The present invention contains following advantages:

1. The method of present invention to prepare micro-particle drug in hemisphere-shaped dosage form is novel and its procedure is simple, fast and easily operated.

2. The micro-particle drug in hemisphere-shaped dosage form prepared by the method of the present invention has a different drug release rate from that of micro-particle drug with spherical, cylindrical, and slab shapes, and the method of the present invention could be applied to any suitable drug.

3. Any drug that is suitable to be prepared by the present invention could reach zero-order drug release rate in patient's body after being produced by this method of the invention.

Many changes and modifications in the above described embodiment of the invention can, of course, be carried out without departing from the scope thereof. Accordingly, to promote progress in science and the useful arts, the invention is disclosed and is intended to be limited only by the scope of the appended claims.

1. A method for preparing a micro-particle drug in hemisphere-shaped dosage form, comprising:
dropping a high molecular weight solution containing drug on a base material to produce a hemisphere-shaped liquid drop of drug solution; and
performing a solidifying process to obtain a solidified micro-particle drug in hemisphere-shaped dosage form,
wherein the solidifying processes comprises at least one of cross-link and evaporation, and
wherein the high molecular weight solution comprises at least one of alginate, chitin, chitosan, poly lactic acid (PLA), or poly glycolic acid (PGA).

2. The method as recited in claim 1, wherein the high molecular weight solution is aqueous and comprises at least 1%˜5% drug, 1%˜40% alginate, and 55%˜98% water wherein the solidifying process is preceded by adding effective amount of a calcium ion, compound or composition that contains calcium into the hemisphere-shaped aqueous drop of drug solution to trigger a cross-link reaction, and a solidified micro-particle of alginate in a hemisphere-shaped dosage form is obtained.

3. The method as recited in claim 1, wherein the high molecular weight solution comprises at least 0.05%˜20% drug, 95%˜49.95% poly lactic acid (PLA), and 50%˜98% organic solvent, wherein the organic solvent comprises at least one of methylene chloride, ethyl acetate, and acetone.

4. The method as recited in claim 1, wherein the high molecular weight solution containing drug comprises at least 0.05%˜20% drug, 1.95%˜49.95% poly lactic acid (PLA), and 50%˜98% methylene chloride, and the solidified micro-particle drug in hemisphere-shaped dosage form comprises 0.1%˜40% drug and 60%˜99.9% PLA after the organic solvent is evaporated completely.

5. A micro-particle drug in hemisphere-shaped dosage form prepared by the method of claim 1.

6. The micro-particle drug in hemisphere-shaped dosage form as recited in claim 5, wherein the size of the said micro-particle is micron-scale.

7-8. (canceled)

9. The micro-particle drug in hemisphere-shaped dosage form as recited in claim 5, wherein the size of the said micro-particle is nano-scale.

10. A micro-particle drug in hemisphere-shaped dosage form, comprising at least 1%˜5% drug, 1%˜40% alginate and 55%˜98% water.

11. A micro-particle drug in hemisphere-shaped dosage form, comprising at least 0.1%˜40% drug and 60%˜99.9% poly lactic acid.
